(19) United States
(12) Patent Application Publication
(10) Pub. No.: US 2010/0047311 A1
(43) Pub. Date: Feb. 25, 2010

(54) PROTECTING ALGAE FROM BODY FLUIDS

(75) Inventors: Avi Rotem, Petach Tikva (IL); Chanan Schneider, Tirat Yehuda (IL); Tova Neufeld, Ariel (IL); Yoav Evron, Hod Hasharon (IL)

Correspondence Address: SUGHRUE MION, PLLC, 2100 PENNSYLVANIA AVENUE, N.W., SUITE 800, WASHINGTON, DC, 20037, US

(73) Assignee: BETA O2 TECHNOLOGIES LTD., Petach Tikva (IL)

(21) Appl. No.: 12/515,818

(22) PCT Filed: Nov. 22, 2007
(86) PCT No.: PCT/IL07/01447

§ 371 Date: Jul. 31, 2009

Related U.S. Application Data
(60) Provisional application No. 60/860,632, filed on Nov. 22, 2006.

(51) Int. Cl. A61F 2/00; A61K 36/02

(52) U.S. Cl. 424/423; 424/195.17

(57) ABSTRACT

Apparatus (20) is provided for implantation into a body of a subject, including isolated functional cells (28). At least one first barrier (22) having a first molecular weight cutoff is disposed with respect to the functional cells so as to protect the functional cells from components disposed within body fluid of the subject having molecular weights higher than the first cutoff. Photosynthetic elements (26) are disposed with respect to the functional cells so as to provide oxygen thereto. At least one second barrier (24) has a second molecular weight cutoff that is lower than the first cutoff. The second barrier is disposed with respect to the photosynthetic elements so as to protect the photosynthetic elements from components disposed within the body fluid of the subject having molecular weights higher than the second cutoff. Other embodiments are also described.

### CROSS-REFERENCES TO RELATED APPLICATIONS

The present application claims priority from US Provisional Patent Application 60/860,632 to Rotem et al., filed Nov. 22, 2006, entitled, “Protecting algae from body fluids,” which is assigned to the assignee of the present invention and is incorporated herein by reference.

### FIELD OF THE INVENTION

The present invention generally relates to implantable medical devices. Specifically, the present invention relates to an implantable device to provide oxygen for isolated functional cells.

### BACKGROUND OF THE INVENTION

Oxygen is an essential component in sustaining implanted isolated cells. A lack of oxygen will lead to cell pathology and ultimately cell death. Oxygen provision is a vital component in sustaining transplanted cells.

U.S. Pat. Nos. 4,352,883, 5,427,935, 5,879,709, 5,902,745, and 5,912,005, which are incorporated herein by reference, describe methods for immunoprotection of biological materials by encapsulation. Encapsulating materials are selected so as to be biocompatible and to allow diffusion of small molecules while shielding the encapsulated cells from immunoglobulins and cells of the immune system. Encapsulated “isolated” functional cells, such as beta cells, for example, can be injected into a vein or embedded under the skin, in the abdominal cavity, or in other locations.

PCT Publication WO 01/50983 to Vardi et al., and U.S. patent application Ser. No. 10/466,069 in the national phase thereof, which are assigned to the assignee of the present application and are incorporated herein by reference, describe an implantable device comprising a chamber for holding functional cells and an oxygen generator for providing oxygen to the functional cells. In one embodiment, the oxygen generator is described as comprising photosynthetic cells that convert carbon dioxide to oxygen when illuminated. In another embodiment, the oxygen generator is described as comprising electrodes that produce oxygen by electrolysis.

US Patent Application Publication 2005/0136092 to Rotem et al., which is assigned to the assignee of the present patent application and is incorporated herein by reference, describes apparatus including a chamber, which is adapted to be implanted in a body of an individual, the chamber including functional cells and chlorophyll-containing elements comprising chlorophyll of an obligate photoautotroph. Typically, the chlorophyll-containing elements include intact photosynthetic cells and/or isolated chloroplasts. The chlorophyll-containing elements provide oxygen to the functional cells and/or consume carbon dioxide produced by the functional cells. The chamber has one or more walls that are adapted to be permeable to nutrients and substances produced or secreted by the cells. The walls also typically immunoisolate the cells from constituents of the body. The chamber is adapted to be implanted under skin of the subject, or in the peritoneum. The apparatus further comprises a light source that is adapted to provide light to the chlorophyll-containing elements.

PCT Publication WO 06/059322 to Evron et al., which is assigned to the assignee of the present patent application and is incorporated herein by reference, describes apparatus including a chamber which is adapted to be implanted in a body of an individual. The chamber includes functional cells and chlorophyll-containing elements comprising chlorophyll of an obligate photoautotroph. Other embodiments are also described.

U.S. Pat. No. 5,713,888 to Neuenfeldt et al., which is incorporated herein by reference, describes an implant assembly for a host tissue. The implant assembly comprises a pouch including wall means defining a chamber for holding a second member. The wall means includes an outer vascularizing membrane having a conformation that results in growth of vascular structures by the host tissue, close to an interface between the vascularizing membrane and host tissue. The assembly includes a second member that can be removably inserted in the chamber, including an interior for receiving cells, and wall means defining an immuno-isolating membrane that isolates the cells from the immune response of the host tissue.

The following patents and patent applications, which are incorporated herein by reference, may be of interest:

U.S. Pat. No. 4,721,677 to Clark, Jr. et al.

U.S. Pat. No. 5,614,378 to Yang et al.

U.S. Pat. No. 6,268,161 to Han, et al.

U.S. Pat. No. 6,368,592 to Colton et al.

U.S. Pat. No. 6,383,478 to Prokop, et al.

U.S. Pat. No. 6,630,154 to Fraker, et al.

U.S. Pat. No. 6,960,351 to Dionne et al.

US Patent Application Publication 2003/0113302 to Revazova et al.

US Patent Application Publication 2005/0025680 to Monzyk et al.

US Patent Application Publication 2006/0024276 to Ricordi et al.

The following articles, which are incorporated herein by reference, may be of interest:

Faithful N S, “Fluorocarbons. Current status and future applications,” Anaesthesia, 42(3):234-242 (1987)

Kaisers U et al., “Liquid ventilation,” British Journal of Anaesthesia 91(1):143-151 (2003)

Lacy P E et al., “Maintenance of normoglycemia in diabetic mice by subcutaneous xenografts of encapsulated islets,” Science 1782-4 (1991)

NASA Tech Briefs MSC-21480, U.S. Govt. Printing Office, Washington, D.C. 20402

Silva A I et al., “An overview on the development of a bio-artificial pancreas as a treatment of insulin-dependent diabetes mellitus,” Med Res Rev 26(2):181-222 (2006)

### SUMMARY OF THE INVENTION

In some embodiments of the present invention, apparatus containing transplanted cells comprises a housing designated for implantation within a body of a subject. Typically, the housing comprises (a) isolated functional cells, e.g., pancreatic islets of Langerhans, and (b) photosynthetic elements. The isolated functional cells and the photosynthetic elements are surrounded by first and second semi-permeable barriers, respectively, which protect the cells and the photosynthetic elements from components disposed within the body fluid of the subject. The first barrier, surrounding the functional cells, has a first molecular weight cutoff, which restricts passage through the barrier of components disposed within the body fluid that are larger than the first cutoff. The second barrier, surrounding the photosynthetic elements, has a second molecular weight cutoff which is lower than the first cutoff, and restricts passage of body components that are larger than the second molecular weight cutoff. Thus, the photosynthetic elements are protected from at least some types of components of the body fluid to which the functional cells are exposed.

Typically, the housing is subcutaneously implanted into the body of the subject. Alternatively, the housing is implanted at another intrabody site.

In an embodiment of the present invention, the photosynthetic elements comprise algae. Alternatively or additionally, the photosynthetic elements comprise isolated chloroplasts and/or photosynthetic organisms. Typically, the photosynthetic elements supply oxygen to the functional cells and consume carbon dioxide produced by the functional cells. The semi-permeable barriers surrounding both the functional cells and the photosynthetic elements are thus gas permeable, facilitating bidirectional passage of gases between the functional cells and the photosynthetic elements.

In some embodiments of the present invention, the second barrier surrounding the photosynthetic elements is surrounded at least in part by the first barrier, which in turn also surrounds the isolated functional cells. In such an embodiment, oxygen is transferred from the photosynthetic elements to the surrounding functional cells. Additionally, such a configuration provides supplemental protection of the photosynthetic elements by both the first and second barriers.

In some embodiments of the present invention, the first barrier housing the functional cells is disposed adjacent to the second barrier housing the photosynthetic elements. Alternatively, different portions of the functional cells are surrounded by respective semi-permeable first barriers, each of which has a molecular weight cutoff as stated hereinabove with respect to the cutoff of the first barrier. Similarly, different portions of the photosynthetic elements are surrounded by respective semi-permeable barriers, each of which has a molecular weight cutoff as stated hereinabove with respect to the cutoff of the second barrier. In this embodiment, the barriers surrounding both the photosynthetic elements and the functional cells are typically but not necessarily generally spherically shaped. Such a configuration of multiple small spheres increases the total surface area, thus facilitating more efficient oxygen transfer between the photosynthetic elements and the functional cells.

There is therefore provided, in accordance with an embodiment of the invention, apparatus for implantation into a body of a subject, including:

isolated functional cells;

at least one first semi-permeable barrier having associated therewith a first molecular weight cutoff, disposed with respect to the functional cells so as to protect the functional cells from components disposed within a body fluid of the subject having molecular weights higher than the first cutoff;

photosynthetic elements, disposed with respect to the functional cells so as to provide oxygen thereto; and

at least one second semi-permeable barrier having associated therewith a second molecular weight cutoff that is lower than the first cutoff, the second barrier disposed with respect to the photosynthetic elements so as to protect the photosynthetic elements from components disposed within the body fluid of the subject having molecular weights higher than the second cutoff.

In an embodiment, the components disposed within the body fluid include antibiotic molecules, and the second semi-permeable barrier is configured to protect the photosynthetic elements from the antibiotic molecules.

In an embodiment, the photosynthetic elements include algae.

In an embodiment, the photosynthetic elements include at least one photosynthetic element selected from the group consisting of: isolated chloroplasts, and photosynthetic organisms.

In an embodiment, the apparatus is configured for subcutaneous implantation in the subject.

In an embodiment, the apparatus includes a light source configured to provide light for the photosynthetic elements.

In an embodiment, the light source includes a light emitting diode (LED).

In an embodiment, the first cutoff is greater than 1000 Daltons.

In an embodiment, the second cutoff is greater than 100 Daltons.

In an embodiment, the second cutoff is greater than 300 Daltons.

In an embodiment, the second cutoff is less than 5000 Daltons.

In an embodiment, the second cutoff is less than 1500 Daltons.

In an embodiment, the first cutoff is greater than two times the second cutoff.

In an embodiment, the first cutoff is greater than 10 times the second cutoff.

In an embodiment, the functional cells produce a desired large molecule having a molecular weight associated therewith, and wherein the molecular weight cutoff of the second barrier is lower than the molecular weight of the large molecule.

In an embodiment, the functional cells are capable of performing at least one of the actions selected from the list consisting of: absorbing a substance from the body, and degrading a substance from the body, and wherein the molecular weight cutoff of the second barrier is lower than a molecular weight of the substance.

In an embodiment, the second cutoff is between 200 and 1000 Daltons.

In an embodiment, the second cutoff is between 300 and 500 Daltons.

In an embodiment, the at least one second semi-permeable barrier includes a hydrophobic semi-permeable barrier.

In an embodiment, the apparatus includes a housing, wherein the first semi-permeable barrier and the second semi-permeable barrier are coupled to the housing.

In an embodiment, the first semi-permeable barrier is shaped to define the housing.

In an embodiment, the first semi-permeable barrier and the second semi-permeable barrier are disposed within the housing.

In an embodiment, the first semi-permeable barrier surrounds a first region of the apparatus, and wherein the second semi-permeable barrier surrounds a second region of the apparatus.

In an embodiment, the first and second barriers are gas permeable.

In an embodiment, the first and second barriers are configured to facilitate bidirectional passage therethrough of gases between the first and second regions.

In an embodiment, the at least one first semi-permeable barrier includes a plurality of first semi-permeable barriers surrounding respective portions of the functional cells, and the at least one second semi-permeable barrier includes a plurality of second semi-permeable barriers surrounding respective portions of the photosynthetic elements.

In an embodiment, the molecular weight cutoffs of each of the first barriers is greater than two times the molecular weight cutoffs of each of the second barriers.

In an embodiment, the molecular weight cutoffs of each of the first barriers is-greater than ten times the molecular weight cutoffs of each of the second barriers.

In an embodiment, the plurality of first semi-permeable barriers are generally spherical.

In an embodiment, the plurality of second semi-permeable barriers are generally spherical.

In an embodiment, a majority of the first semi-permeable barriers are in contact with at least one other one of the first or second barriers.

In an embodiment, a majority of the first semi-permeable barriers are not in contact with at least one other one of the first or second barriers.

There is further provided, in accordance with an embodiment of the invention, a method, including:

protecting isolated functional cells from a first set of components disposed within body fluid of a subject by using at least one first semi-permeable barrier having associated therewith a first molecular weight cutoff;

protecting photosynthetic elements from a second set of components disposed within the body fluid of the subject by using at least one second semi-permeable barrier having associated therewith a second molecular weight cutoff, the first molecular weight cutoff being lower than the first cutoff; and

implanting within the body of the subject the first semi-permeable barrier, the second semi-permeable barrier, the functional cells, and the photosynthetic elements.

The present invention will be more fully understood from the following detailed-description of embodiments thereof, taken together with the drawings, in which:

### BRIEF DESCRIPTION OF THE DRAWINGS

FIG. 1 is a schematic illustration of a housing coupled to functional cells and photosynthetic elements, in accordance with an embodiment of the present invention;

FIG. 2 is a schematic illustration of housing coupled to functional cells and photosynthetic elements, in accordance with another embodiment of the present invention;

FIG. 3 is a schematic illustration of a housing coupled to functional cells and photosynthetic elements, in accordance with yet another embodiment of the present invention;

FIG. 4 is a schematic illustration of photosynthetic elements surrounded by a semi-permeable barrier, in accordance with an embodiment of the present invention;

FIG. 5 is a schematic illustration of photosynthetic elements surrounded by a gas permeable barrier, in accordance with an embodiment of the present invention; and

FIG. 6 is a graph showing oxygen production by photosynthetic elements, in accordance with an embodiment of the present invention.

### DETAILED DESCRIPTION OF EMBODIMENTS

Reference is now made to FIG. 1, which is a schematic illustration of apparatus 20 comprising a housing 18 comprising a first semi-permeable barrier 22 and a second semi-permeable barrier 24 configured for implantation into a body of a subject, in accordance with an embodiment of the present invention. Typically, but not necessarily, apparatus 20 is designated for subcutaneous implantation. Functional cells 28 are disposed within a first region of apparatus 20, the first region being surrounded by first semi-permeable barrier 22. Photosynthetic elements 26 are disposed within a second region of apparatus 20, the second region being surrounded by second semi-permeable barrier 24. The functional cells and photosynthetic elements are typically disposed within a matrix, which itself comprises, for example, a semi-permeable polymeric substance such as: agar, agarose, alginate, polyethylene glycol and chitosan. Typically, first semi-permeable barrier 22 immunoisolates functional cells 28 from components such as white blood cells that are disposed within the body fluid of the subject. Second semi-permeable barrier 24 immunoisolates photosynthetic elements 26 from white blood cells, and, additionally, protects photosynthetic elements 26 from components (e.g., antibiotics or other natural or artificial small molecules) disposed within the body fluid of the subject, while allowing passage of very small molecules such as water, oxygen, and carbon dioxide.

Second barrier 24 is disposed within the first region, e.g., in the center of apparatus 20, or near an edge thereof. Functional cells 28 are disposed around barrier 24, and are, in turn, surrounded by first barrier 22. Such a configuration of first barrier 22 with respect to second barrier 24 provides supplemental protection of photosynthetic elements 26 by first barrier 22 from the components of the body fluid of the subject.

First semi-permeable barrier 22 is typically permeable to molecules that are larger than the molecules allowed passage through second semi-permeable barrier 24. First semi-permeable barrier 22 has a molecular weight cutoff that is typically higher than the molecular weight cutoff of second semi-permeable barrier 24. The relatively higher molecular weight cutoff of first barrier 22 allows passage into the first region of components (e.g., nutrients) disposed within the body fluid of the subject. Additionally, first semi-permeable barrier 22 is configured to allow passage therethrough of large molecules, e.g., insulin, produced by the functional cells. The molecular weight cutoff of second semi-permeable barrier 24 restricts passage therethrough of larger molecules which are allowed passage through first barrier 22. For example, the molecules produced by functional cells 26 typically have a molecular weight that is smaller than the cutoff of first barrier 22 (and thus are allowed passage out of the first region through barrier 22), and larger than the cutoff of second barrier 24 (and thus are restricted from passing into the second region). In some embodiments, first barrier 22 has a molecular weight cutoff greater than two times the molecular weight cutoff of second barrier 24. For example, first barrier 22 may have a molecular weight cutoff greater than ten times the molecular weight cutoff of second barrier 24.

In some embodiments, first barrier 22 has a cutoff greater than 1000 Daltons. In an embodiment, second barrier 24 has a cutoff greater than 100 Daltons, e.g., 300 Daltons. In an embodiment, second barrier 24 has a molecular weight cutoff less than 5000 Daltons, e.g., less than 1500 Daltons. In some embodiments, second semi-permeable barrier 24 is permeable to molecules having a molecular weight between 200 and 1000 Daltons, e.g., between 300 and 500 Daltons.

For some applications, functional cells 28 disposed within first semi-permeable barrier 22 are capable of absorbing a substance from the body, and/or degrading a substance from the body. The molecular weight of the substance is lower than the molecular weight cutoff of first barrier 22, and is typically higher than the molecular weight cutoff of second barrier 24.

A light source (e.g., a light emitting diode) provides light energy to facilitate photosynthesis by photosynthetic elements 26. Photosynthetic elements 26 (e.g., algae, isolated chloroplasts, and/or photosynthetic organisms) produce and supply oxygen to functional cells 28. Second semi-permeable barrier 24 is typically gas-permeable so as to facilitate bidirectional flow of gases between photosynthetic elements 26 and functional cells 28.

In some embodiments, second semi-permeable barrier 24 is a hydrophobic semi-permeable barrier, e.g., in order to enable rapid oxygen diffusion therethrough.

In some embodiments, apparatus 20 comprises a third barrier (not shown, or constituting the matrix in which the cells and photosynthetic elements are disposed), which typically surrounds first barrier 22, thereby providing an additional layer of protection for components of apparatus 20. In some embodiments, the third semi-permeable barrier has a molecular weight cutoff higher than the molecular weight cutoff of first barrier 22 and higher than the molecular weight cutoff of the second barrier 24. The third semi-permeable barrier is configured to provide supplemental protection to functional cells 28 and to photosynthetic elements 26. Alternatively, the third semi-permeable barrier has a molecular weight cutoff substantially equal to the molecular weight cutoff of first barrier 22, and higher than the molecular weight cutoff of second semi-permeable barrier 24.

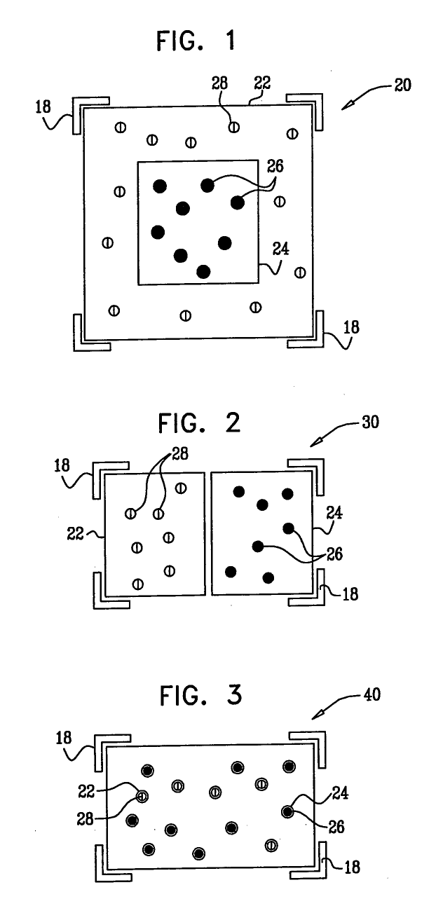

Reference is now made to FIG. 2, which is a schematic illustration of apparatus 30, comprising housing 18, which in turn comprises first semi-permeable barrier 22 and second semi-permeable barrier 24 disposed adjacently thereto, in accordance with an embodiment of the present invention. Apparatus 30 is generally similar to apparatus 20, as described hereinabove with reference to FIG. 1, except for differences as described hereinbelow. As described hereinabove, functional cells 28 are surrounded by first semi-permeable barrier 22 which defines the first region of apparatus 30, while photosynthetic elements 26 are surrounded by semi-permeable barrier 24 which defines the second region of the housing of apparatus 30. At least a portion of first semi-permeable barrier 22 and at least a portion of second semi-permeable barrier 24 are gas-permeable to facilitate the bidirectional flow of gases between functional cells 28 and photosynthetic elements 26. These portions typically define the adjacent portions of barriers 22 and 24.

In some embodiments, apparatus 30 is surrounded by a third semi-permeable barrier (not shown), which is described hereinabove with reference to FIG. 1. The third barrier surrounds both barriers 22 and 24 and the respective first and second regions defined thereby.

Reference is now made to FIG. 3, which is a schematic illustration of apparatus 40, comprising a housing comprising a plurality of first and second barriers 22 and 24, in accordance with an embodiment of the present invention. Apparatus 40 is generally similar to apparatus 20 and apparatus 30, as described hereinabove with reference to FIGS. 1 and 2, except for differences as described hereinbelow.

Respective portions of functional cells 28 are surrounded by a plurality of first semi-permeable barriers 22, and respective portions of photosynthetic elements 26 are surrounded by a plurality of second semi-permeable barriers 24. Barriers 22 surrounding functional cells 28 are dispersed among barriers 24 surrounding photosynthetic elements 26. The relative positioning of barriers 22 and 24 reduces the distance between the functional cells 28 and the photosynthetic elements 26, thereby increasing the efficiency of the transfer of oxygen from photosynthetic elements 26 to functional cells 28. Additionally, barriers 22 and 24 are typically generally spherically shaped. The presence of many small spheres of barriers 22 and 24 surrounding their respective contents increases the total effective surface area of barriers 22 and barriers 24, thereby increasing the efficiency of oxygen transfer between photosynthetic elements 26 and functional cells 28.

In some embodiments of the present invention, barriers 22 and 24 are generally not in contact with one another (as shown). In some embodiments, a majority of barriers 22 are in contact with at least one of the plurality of barriers 24 and/or with another one of the plurality of barriers 22.

In some embodiments, apparatus 40 is surrounded by a third semi-permeable barrier (not shown), which is described hereinabove with reference to FIG. 1. In some embodiments, the third barrier defines housing 18 of apparatus 40. The third barrier provides an additional layer of protection for both functional cells 28 and photosynthetic elements 26 from components disposed within the body fluid of the subject.

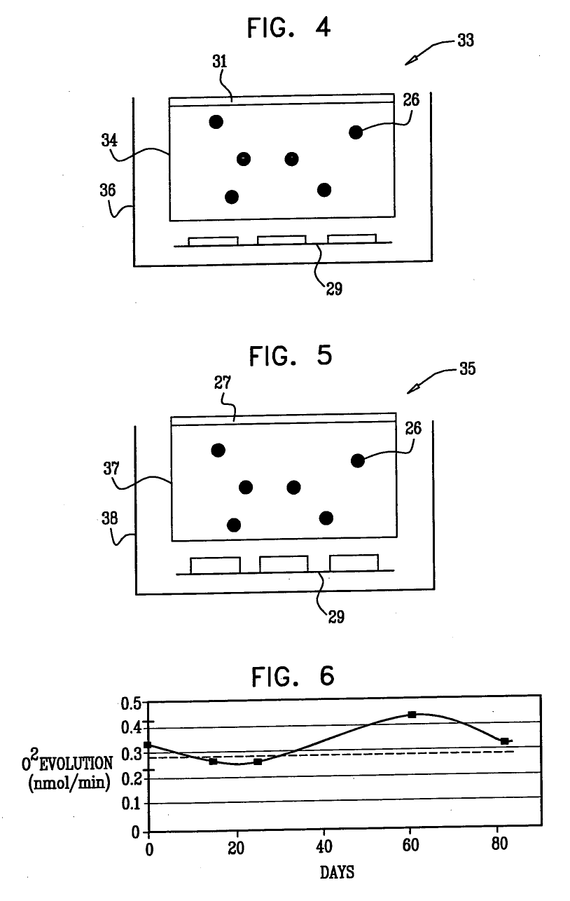

FIGS. 4-5 are schematic illustrations of systems 33 and 35 used in two experiments conducted to assess the respective protective abilities of a liquid-permeable barrier 31 and a gas-permeable barrier 27, in accordance with an embodiment of the present invention.

During the first experiment, schematically illustrated in FIG. 4, photosynthetic elements 26 comprising algae were placed within a chamber 34 of a silicone-rubber housing 36. Barrier 31 comprised a Spectropore cellulose-ester barrier having a molecular weight cutoff of 100 Daltons.

Photosynthetic elements 26 were concentrated by centrifugation, mixed with 1.5% or 2% alginate, and subsequently placed within chamber 34 of housing 36. The alginate immobilized photosynthetic elements 26 once they were disposed within chamber 34.

Housing 36 was coupled to an LED array light source 29, and implanted into the back of a rat (“the experimental rat”). A portion of chamber 34 was exposed to the body fluids of through rat, through semi-permeable barrier 31. The alginate in which photosynthetic elements 26 were disposed prevented contact between the photosynthetic elements and white blood cells of the rat.

Additionally, a similar system serving as a control (not shown) was implanted within the body of a control rat. This control system comprised a silicone-rubber housing without semi-permeable barrier 31. Photosynthetic elements 26 comprising algae, prepared according to the specifications described hereinabove, were injected within the housing, which was subsequently implanted within the body of the rat. The photosynthetic elements were disposed in the housing within an alginate matrix, which immunoisolated the elements from white blood cells.

In the control rat, photosynthetic elements 26 were destroyed within five days following implantation. The inventors hypothesize that this is because photosynthetic elements 26 in the control rat were not protected from the components of the body fluid of the rat that were blocked by barrier 31 in the experimental rat.

Housing 36 of system 33 was extracted from within the body of the experimental rat following a period of one month. The oxygen concentration of system 33 was measured using a Clack-type oxygen electrode. In contrast to the control rat, photosynthetic elements of system 33 were capable of oxygen production even after a period of one month.

During the second experiment, schematically illustrated in FIG. 5, photosynthetic elements 26 comprising algae were placed into a chamber 37 of a housing 38. A portion of chamber 37 was designated to be exposed to body fluids of a rat through liquid-impermeable, gas-permeable barrier 27, in this case a silicone/Teflon membrane. Barrier 27 in this experiment had a width of 25 um.

Photosynthetic elements 26 were concentrated by centrifugation, mixed with liquid agarose, and subsequently injected into chamber 37 of housing 38. The agarose immobilized photosynthetic elements 26 once they were disposed within chamber 37.

Housing 38 was coupled to an LED array light source 29 and implanted into the back of a rat.

FIG. 6 is a graph showing the evolution of oxygen production of system 35, as recorded in accordance with an embodiment of the present invention. On days 0 and 80 and three intervening days, the rate of oxygen production of system 35 was tested by measuring using a Clack-type oxygen electrode the amount of oxygen produced by the photosynthetic elements of system 35. Results demonstrate the ability of gas permeable barrier 27 to protect photosynthetic elements 26 in the body for a period of at least 80 days.

It is to be noted that the scope of the present invention includes the use of implantable oxygen generators, e.g., as described in U.S. Pat. No. 6,368,592 to Colton et al., U.S. Pat. No. 6,960,351 to Dionne et al., and/or U.S. Pat. No. 4,721,677 to Clark, Jr. et al., which are incorporated herein by reference. Use of such oxygen generators is configured to provide supplemental oxygen production for the isolated functional cells in combination with the oxygen produced by photosynthetic elements 26 described herein.

The scope of the present invention includes embodiments described in one or more of the following:
PCT Patent Application PCT/IL01/00031 to Vardi et al., entitled, “Implantable device,” filed Jan. 12, 2001;
U.S. patent application Ser. No. 10/466,069 to Vardi et al., entitled, “Implantable device,” filed Mar. 12, 2004;
U.S. patent application Ser. No. 11/001,556 to Rotem et al., entitled “Implantable device,” filed Nov. 30, 2004;
PCT Patent Application PCT/IL2005/001262 to Evron et al., entitled, “Implantable device,” filed Nov. 27, 2005;
U.S. Provisional Patent Application 60/860,632 to Rotem et al., entitled, “Protecting algae from body fluids,” filed Nov. 22, 2006, which is assigned to the assignee of the present patent application and is incorporated herein by reference. For some applications, techniques described in that provisional patent application are performed in combination with techniques described herein;
U.S. Provisional Patent Application 60/861,592 to Rotem et al., entitled, “Oxygen supply for cell transplant and vascularization,” filed Nov. 28, 2006; and
a US provisional patent application, entitled “Air gaps for supporting cells,” to Rozy et al., filed Sep. 7, 2007.
   All of these applications are incorporated herein by reference.

For some applications, techniques described herein are practiced in combination with techniques described in one or more of the entries in the above list or in references cited in the Cross-references section or Background section of the present patent application, which are incorporated herein by reference.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for implantation into a body of a subject, comprising:
isolated functional cells;
at least one first semi-permeable barrier having associated therewith a first molecular weight cutoff, disposed with respect to the functional cells so as to protect the functional cells from components disposed within a body fluid of the subject having molecular weights higher than the first cutoff;
photosynthetic elements, disposed with respect to the functional cells so as to provide oxygen thereto; and
at least one second semi-permeable barrier having associated therewith a second molecular weight cutoff that is lower than the first cutoff, the second barrier disposed with respect to the photosynthetic elements so as to protect the photosynthetic elements from components disposed within the body fluid of the subject having molecular weights higher than the second cutoff.

2. The apparatus according to claim 1, wherein the components disposed within the body fluid include antibiotic molecules, and wherein the second semi-permeable barrier is configured to protect the photosynthetic elements from the antibiotic molecules.

3. The apparatus according to claim 1, wherein the photosynthetic elements comprise algae.

4. The apparatus according to claim 1, wherein the photosynthetic elements comprise at least one photosynthetic element selected from the group consisting of: isolated chloroplasts, and photosynthetic organisms.

5. The apparatus according to claim 1, wherein the apparatus is configured for subcutaneous implantation in the subject.

6. The apparatus according to claim 1, further comprising a light source configured to provide light for the photosynthetic elements.

7. The apparatus according to claim 6, wherein the light source comprises a light emitting diode (LED).

8. The apparatus according to claim 1, wherein the first cutoff is greater than 1000 Daltons.

9. The apparatus according to claim 1, wherein the second cutoff is greater than 100 Daltons.

10. The apparatus according to claim 1, wherein the second cutoff is greater than 300 Daltons.

11. The apparatus according to claim 1, wherein the second cutoff is less than 5000 Daltons.

12. The apparatus according to claim 1, wherein the second cutoff is less than 1500 Daltons.

13. The apparatus according to claim 1, wherein the first cutoff is greater than two times the second cutoff.

14. The apparatus according to claim 1, wherein the first cutoff is greater than 10 times the second cutoff.

15. The apparatus according to claim 1, wherein the functional cells produce a desired large molecule having a molecular weight associated therewith, and wherein the molecular weight cutoff of the second barrier is lower than the molecular weight of the large molecule.

16. The apparatus according to claim 1, wherein the functional cells are capable of performing at least one of the actions selected from the list consisting of: absorbing a substance from the body, and degrading a substance from the body, and wherein the molecular weight cutoff of the second barrier is lower than a molecular weight of the substance.

17. The apparatus according to claim 1, wherein the second cutoff is between 200 and 1000 Daltons.

18. The apparatus according to claim 1, wherein the second cutoff is between 300 and 500 Daltons.

19. The apparatus according to claim 1, wherein the at least one second semi-permeable barrier comprises a hydrophobic semi-permeable barrier.

20. The apparatus according to claim 1, further comprising a housing, wherein the first semi-permeable barrier and the second semi-permeable barrier are coupled to the housing.

21. The apparatus according to claim 20, wherein the first semi-permeable barrier is shaped to define the housing.

22. The apparatus according to claim 20, wherein the first semi-permeable barrier and the second semi-permeable barrier are disposed within the housing.

23. The apparatus according to claim 1, wherein the first semi-permeable barrier surrounds a first region of the apparatus, and wherein the second semi-permeable barrier surrounds a second region of the apparatus.

24. The apparatus according to claim 23, wherein the first and second barriers are gas permeable.

25. The apparatus according to claim 24, wherein the first and second barriers are configured to facilitate bidirectional passage therethrough of gases between the first and second regions.

26. The apparatus according to claim 1, wherein the at least one first semi-permeable barrier comprises a plurality of first semi-permeable barriers surrounding respective portions of the functional cells, and wherein the at least one second semi-permeable barrier comprises a plurality of second semi-permeable barriers surrounding respective portions of the photosynthetic elements.

27. The apparatus according to claim 26, wherein the molecular weight cutoffs of each of the first barriers is greater than two times the molecular weight cutoffs of each of the second barriers.

28. The apparatus according to claim 26, wherein the molecular weight cutoffs of each of the first barriers is greater than ten times the molecular weight cutoffs of each of the second barriers.

29. The apparatus according to claim 26, wherein the plurality of first semi-permeable barriers are generally spherical.

30. The apparatus according to claim 26, wherein the plurality of second semi-permeable barriers are generally spherical.

31. The apparatus according to claim 26, wherein a majority of the first semi-permeable barriers are in contact with at least one other one of the first or second barriers.

32. The apparatus according to claim 26, wherein a majority of the first semi-permeable barriers are not in contact with at least one other one of the first or second barriers.

33. A method, comprising:
protecting isolated functional cells from a first set of components disposed within body fluid of a subject by using at least one first semi-permeable barrier having associated therewith a first molecular weight cutoff;
protecting photosynthetic elements from a second set of components disposed within the body fluid of the subject by using at least one second semi-permeable barrier having associated therewith a second molecular weight cutoff, the first molecular weight cutoff being lower than the first cutoff; and
implanting within the body of the subject the first semi-permeable barrier, the second semi-permeable barrier, the functional cells, and the photosynthetic elements.

34. The method according to claim 33, wherein implanting comprises subcutaneously implanting the first semi-permeable barrier, the second semi-permeable barrier, the functional cells, and the photosynthetic elements.

35. The method according to claim 33, wherein the molecular weight cutoff of the first barrier is at least 1000 Daltons, and wherein providing the first barrier comprises providing the first barrier having the cutoff of at least 1000 Daltons.

36. The method according to claim 33, wherein the molecular weight cutoff of the second barrier is at least 100 Daltons, and wherein providing the second barrier comprises providing the second barrier having the cutoff of at least 100 Daltons.

37. The method according to claim 33, wherein the molecular weight cutoff of the second barrier is at least 300 Daltons, and wherein providing the second barrier comprises providing the second barrier having the cutoff of at least 300 Daltons.

38. The method according to claim 33, wherein the molecular weight cutoff of the second barrier is less than 1500 Daltons, and wherein providing the second barrier comprises providing the second barrier having the cutoff of less than 1500 Daltons.

39. The method according to claim 33, wherein the molecular weight cutoff of the second barrier is less than 5000 Daltons, and wherein providing the second barrier comprises providing the second barrier having the cutoff of less than 5000 Daltons.

40. The method according to claim 33, wherein the molecular weight cutoff of the second barrier is between 200 and 1000 Daltons, and wherein providing the second barrier comprises providing the second barrier having the cutoff of between 200 and 1000 Daltons.

41. The method according to claim 33, wherein the molecular weight cutoff of the second barrier is between 300 and 500 Daltons, and wherein the second barrier comprises providing the second barrier having the cutoff of between 300 and 500 Daltons.

42. The method according to claim 33, wherein the molecular weight cutoff of the first barrier is greater than two times the molecular weight cutoff of the second barrier, and wherein providing the first barrier comprises providing the first barrier having the cutoff greater than two times the cutoff of the second barrier.

43. The method according to claim 33, wherein the molecular weight cutoff of the first barrier is greater than ten times the molecular weight cutoff of the second barrier, and wherein providing the first barrier comprises providing the first barrier having the cutoff greater than ten times the cutoff of the second barrier.

44. The method according to claim 33, further comprising facilitating bidirectional passage of gases between the isolated functional cells and the photosynthetic elements.

45. The method according to claim 33, wherein protecting the photosynthetic elements comprises restricting passage through the second semi-permeable barrier of a portion of the body fluid that has passed through the first semi-permeable barrier.

46. The method according to claim 33, wherein:
protecting the isolated functional cells comprises providing a plurality of first semi-permeable barriers, the plurality of first semi-permeable barriers providing protection to the isolated functional cells by surrounding respective portions thereof, and
protecting the photosynthetic elements comprises providing a plurality of second semi-permeable barriers, the plurality of second semi-permeable barriers providing protection to the photosynthetic elements by surrounding respective portions thereof.

47. The method according to claim 33, wherein protecting the photosynthetic elements comprises restricting passage, through the second barrier, of a molecule produced by the functional cells, and wherein the molecular weight cutoff of the second barrier is lower than the molecular weight of the molecules produced by the functional cells.

48. The method according to claim 33, wherein protecting the photosynthetic elements comprises restricting passage, through the second barrier, of a molecule produced by the body and absorbable or degradable by the functional cells, and wherein the molecular weight of the molecule is lower than the molecular weight cutoff of the first barrier and higher than the molecular weigh cutoff of the second barrier.
